(19) United States
(12) Patent Application Publication
(10) Pub. No.: US 2007/0005091 A1
(43) Pub. Date: Jan. 4, 2007

(54) Infra-epidermic subcision device for blunt dissection of sub-epidermic tissues

(76) Inventors: Filiberto P. Zadini, North Hills, CA (US); Giorgio C. Zadini, Camarillo, CA (US)

Correspondence Address: FILIBERTO P. ZADINI, 2237 HILLTOP LANE, CAMARILLO, CA, 93012, US

(21) Appl. No.: 11/169,030

(22) Filed: Jun. 27, 2005

Related U.S. Application Data
(63) Continuation-in-part of application No. 11/112,996, filed on Apr. 21, 2005.

(51) Int. Cl. A61M 29/00

(52) U.S. Cl. 606/192

(57) ABSTRACT

A dermatological device for subcision of sub-epidermic tissues. The device is provided with a blunt dermis contacting surface enabling the operator to lift or cause traction to the skin from underneath the skin, after placement of the dermal contacting surface of the device under the skin. By mere skin lifting from underneath, fibrous bands present within the dermis are detached/disrupted/dissected from their attachments to the skin or from their attachments to deeper layers. Detachment/disruption/dissection of the fibrous bands can be aided by the adjunct of a dissecting arm which by rotation can enhance detachment/disruption/dissection of the fibrous bands. Pathological skin conditions such as edematous-fibrosclerotic panniculopathy known also as cellulite or any depressed scar or deep wrinkle can benefit from the device as dissection of the fibrous bands. which cause depression of skin areas. restitutes a nearly anatomical evened up skin surface.

### RELATED CASES

This application is a Continuation-in-Part of our copeding patent application Ser. No. 11/112,996 filed on Apr. 21, 2005 and now pending.

### FIELD OF THE INVENTION

This invention relates to medical apparatuses and methods for treatment of dermatological pathological conditions, such as the edematous-fibrosclerotic panniculopathy, commonly known as cellulite, acne scars or even physiological conditions such as deep wrinkles.

### BACKGROUND—DESCRIPTION OF THE PRIOR ART

Numerous treatments have been devised for the dermatological condition edematous-fibrosclerotic panniculopathy, commonly known as cellulite.

Some of these treatments have a scientific base, some have a pseudo-scientific, empiric base.

The edematous-fibrosclerotic panniculopathy commonly named cellulite, a non medical term coined in Europe, is a disorder of the skin and subcutaneous tissue. The edematous-fibrosclerotic panniculopathy is due to the formation of an abnormal fibrous network in the hypoderm. The abnormal fibrous network encapsulates conglomerates of fat cells causing a subcutaneous architectural disruption which results in a dimples and nodules appearance of the skin, known as orange peel skin. Strands of fibrous tissue connect the skin to deeper tissue layers and also separate compartments that contain conglomerates of fat cells. Cellulite affects more commonly the hips, thighs, glutei, abdominal wall and upper arms. Women are commonly more affected than men. Researchers agree that most of cellulite “cures” have been ineffective. Recent researches have confirmed that cellulite is product of faulty anatomy, genes and hormones.

Anticellulite products with unsubstantiated claims of successful treatment of the condition include creams and gels, brushes, rollers, body wraps toning lotions, electrical stimulation devices, vibrating machines, inflatable hip-high pressurized boots, hormone or enzymes injections and many others.

More recently, radio frequency and laser devices, cold-laser massage devices, combined radio frequency/infrared devices, fat melting injections, targeted liposuction, tissue fillers have been used for the treatment of cellulite with minimal or marginal success, eventually with only transitory improvements.

A more recently devised surgical procedure called skin subcision has shown some promising results. The procedure consists of cutting the cellulitic fibrous bands, the tethers which cause the depression in the skin with a special needle having surgical scalpel-like tip. The dimples, freed from their fibrous attachments, pop up and the skin is able to regain the even, pre-cellulitic aspect. Regretfully, the procedure is not void of complications. Pain, bruises, hemosiderosis have been associated with the procedure as reported in the International Journal of Dermatology, Volume 39 Issue 7, Page 539, July 2000.

### BRIEF SUMMARY OF THE INVENTION

With the present invention, applicants propose a simple medical-surgical device having the scientific prerequisites of being capable of detaching the fibrous attachments that connect the skin to the deeper layers and cause the typical dimples of a cellulitic skin, via blunt dissection, rather than via sharp dissection as currently in use. Detachment of such fibrous attachments resolves the skin dimples, restituting normal appearance to the skin, minimizing complications more likely to develop with the current technique of sharp dissection.

The device is composed of a needle having an expandable balloon in proximity of the tip, connected to a syringe provided with a handle. The operator inserts the needle into the skin, inflates the balloon, grossly shaped as a donut. The balloon once inflated has the double finction of dissecting by outward radial expansion the fibrous bands network and of serving as anchoring device for skin traction purposes. The operator gently pulls up the needle acting upon the syringe handle connected to the needle carrying the expanded balloon. In doing so the operator elevates the skin, stretching it to the point of rupture the cellulitic fibrous bands which cause the dimpling of the skin.

The detachment of the fibrous bands occur by blunt dissection. It is expected that the extensible surrounding blood vessels are just stretched and not severed as in the above mentioned sharp subcision technique. Surrounding structures will be less traumatized being not sharply cut as in the sharp subcision technique. It is reasonable to say that less trauma to the tissue is expected to occur with greater patient comfort and with expectation of lesser complications. In the instances that blunt dissection results insufficient to detach such fibrous bands the device will be able to place the fibrous bands under sufficient tensile traction to be easily detectable at touch by a blindly exploring sharp cutting tool, with consequent economical and efficient severing of such fibrous bands by such cutting tool, or to enable a cutting tool mounted on the skin traction apparatus to economically and efficiently sever such fibrous bands, in both cases with minimal damage to surrounding tissues in a fashion comparable to blunt dissection technique.

### OBJECT OF THE PRESENT INVENTION

It is an object of the present invention to provide a simple, rapidly deployable medical device for the treatment of cellulite, the treatment being based on solid anatomic-pathological foundations.

It is an object of the present invention to provide the consumer with a simple minimally invasive effective, rapidly deployable means and method for improving cosmetic appearance of the skin affected by cellulite.

It is an object of the present invention to provide a safe, simple and effective apparatus and method to target and to induce mechanical lysis of the fibrous bands which are at the core of the formation and persistence of the cellulite in body areas of patient's concern.

It is an object of the present invention to provide the operator with an alternative improved apparatus and method of an already proven effective method of cellulite treatment, i.e. skin subcision: the dissection of the cellulitic fibrous bands. The proposed device dissects the fibrous tissue by blunt, not sharp, dissection, causing less trauma, less bleeding, ultimately less inflammatory reaction in the subcutaneous tissue.

It is an objective of the present invention to provide the operator with a device which by minimal, needle-like skin invasion ensues an adequate traction on the skin from below without any further puncturing or cutting of the skin.

It is an objective of this invention to provide the operator with an adequate skin traction device requiring a single skin hole of a diameter comparable to a needle-like element capable of exerting traction pressure to the skin from below without any further puncturing or cutting of the skin.

It is further objective of this invention to provide the operator with a minimally invasive device for traction of the skin from below to ensue enough tensile traction on fibrous bands responsible of the skin dimpling characterizing the cellulite to cause detachment of such fibrous bands from their attachments to the skin or to deeper layers, and/or to cause easy detection at touch by a blindly exploring sharp cutting tool as a result of the tensile traction applied to them, with consequent economical and efficient severing of such fibrous bands by such cutting tool, or to enable a cutting tool mounted on the skin traction apparatus to economically and efficiently sever such fibrous bands, in both cases with minimal damage to surrounding tissues in a fashion comparable to the blunt dissection technique.

### DRAWING FIGURES

FIG. 1 is a side view of device with the balloon deflated at rest prior to use.

FIG. 2 is a side view of the device with the balloon inflated.

FIG. 3 is as side view of the same device with a larger balloon fully inflated.

FIG. 4 is across sectional view of a detail of the device of FIG. 2 to 3 specifically the inflatable member or balloon or bluntly dissecting member or anchoring member inflated.

FIG. 5 shows a detail of the device specifically the locking mechanism for the plunger of device prior to actuation of the locking mechanism.

FIG. 6 shows a cross sectional view of the skin of a patient with the device in action with the balloon deployed pulled upward by the operator resulting in blunt dissection/disruption of the cellulitic fibrous bands at the skin attachment and or at the deeper layer attachment.

FIG. 7 is a side view of another embodiment of the device illustrated in FIG. 1-6

FIG. 7A is side view of an enlargement of a detail of the device of FIG. 7

FIG. 7B is an enlarged cross section view of a detail of the device of FIG. 7.

FIG. 7C is an enlarged cross section view of a detail of the device of FIG. 7

FIG. 8 is side view of another embodiment of the device illustrated in FIG. 1-6

FIG. 8A is side view of an enlargement of a detail of the device of FIG. 8

FIG. 9 is a side view of another embodiment of the device of FIG. 1-6.

FIG. 9 A is side view of an enlargement of a detail of the device of FIG. 9

FIG. 10 is a side view of another embodiment of the device of FIG. 1-6.

FIG. 10A is a side view of another embodiment of the device of FIG. 10

FIG. 11 is a side view of another embodiment of the device of FIG. 1-6.

FIG. 12 is side view of another embodiment of the device of FIG. 1-6

FIG. 12A is side view of an enlargement of a detail of the device of FIG. 12

FIG. 13 is a side view of another embodiment of the device of FIG. 1-6.

FIG. 13A is side view of an enlargement of a detail of the device of FIG. 13

FIG. 13B is side view of an enlargement of a detail of the device of FIG. 13

FIG. 14 is a perspective view of another embodiment the device illustrated in FIG. 11.

FIG. 15 is a perspective view of a component of embodiment of FIG. 14

FIG. 16 is a perspective view of another component of embodiment of FIG. 14.

FIG. 17 is a perspective view of the embodiment of FIG. 14 in a stage of deployment.

### DETAILED DESCRIPTION OF THE INVENTION

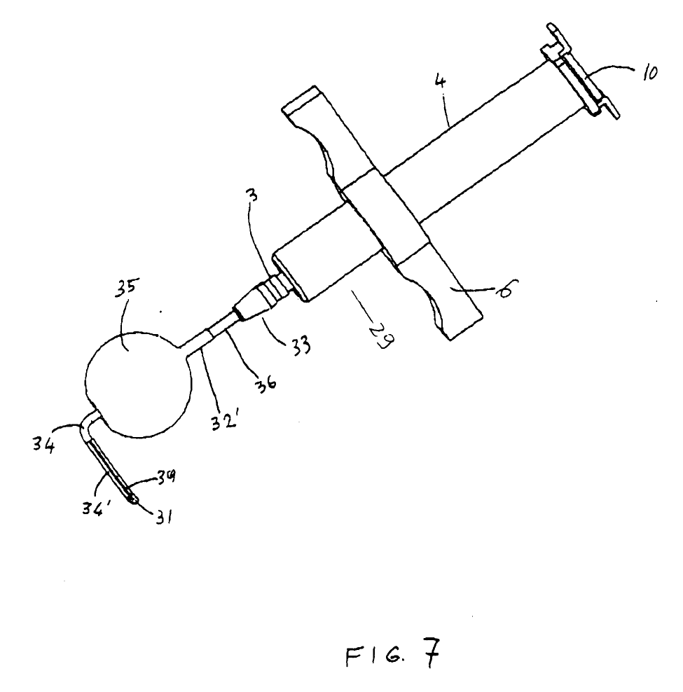

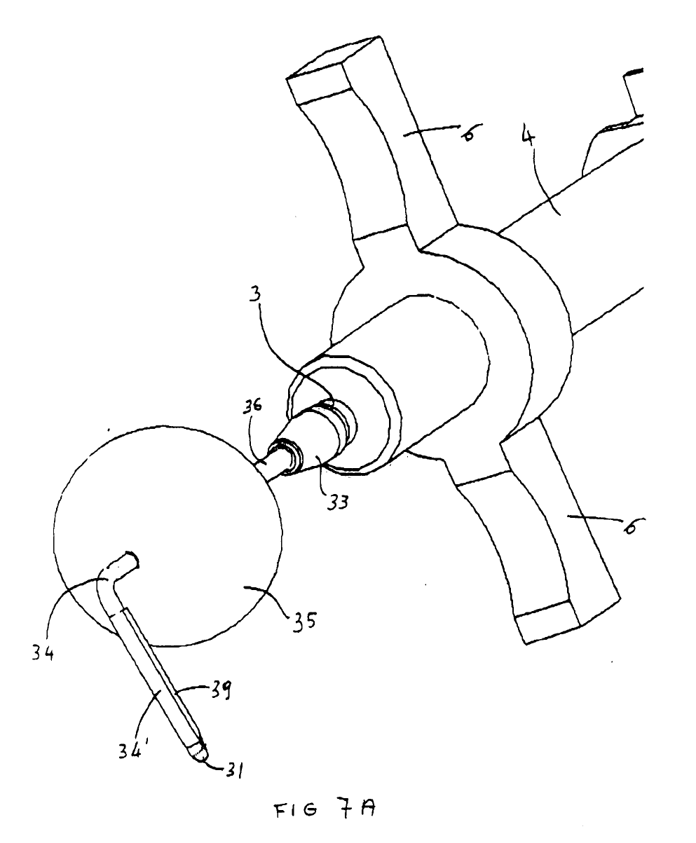

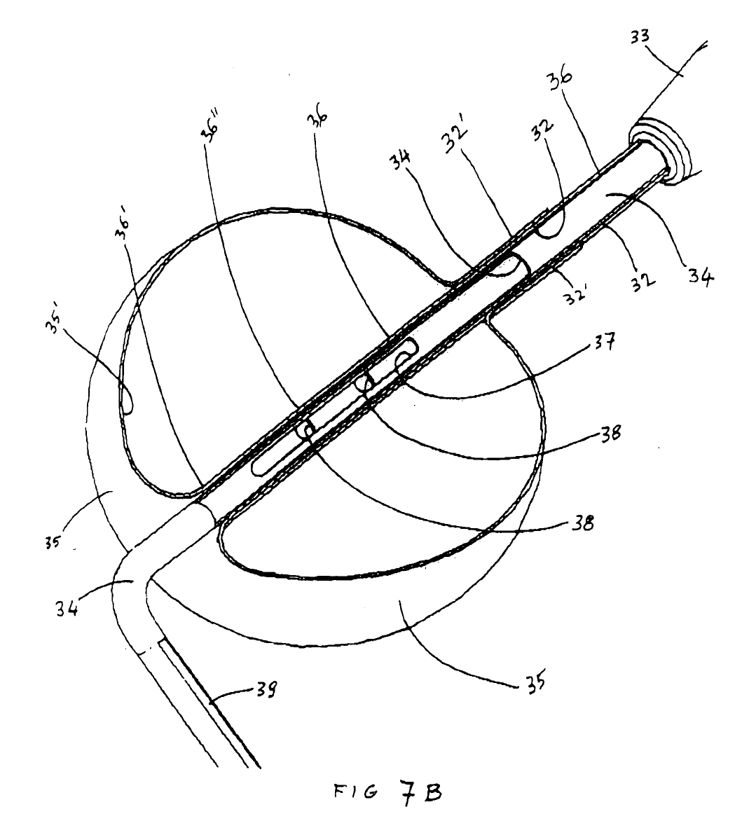

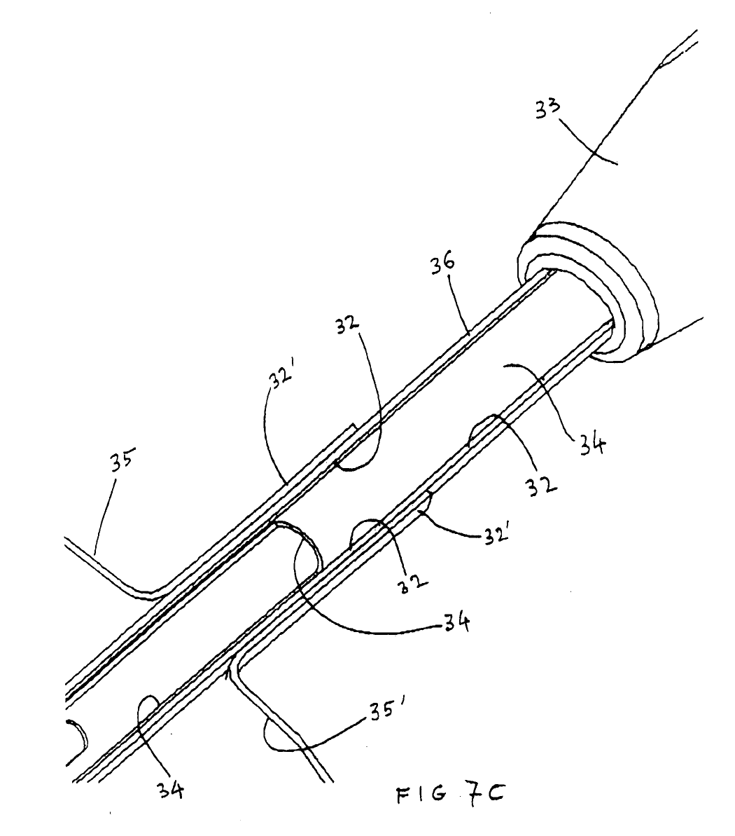

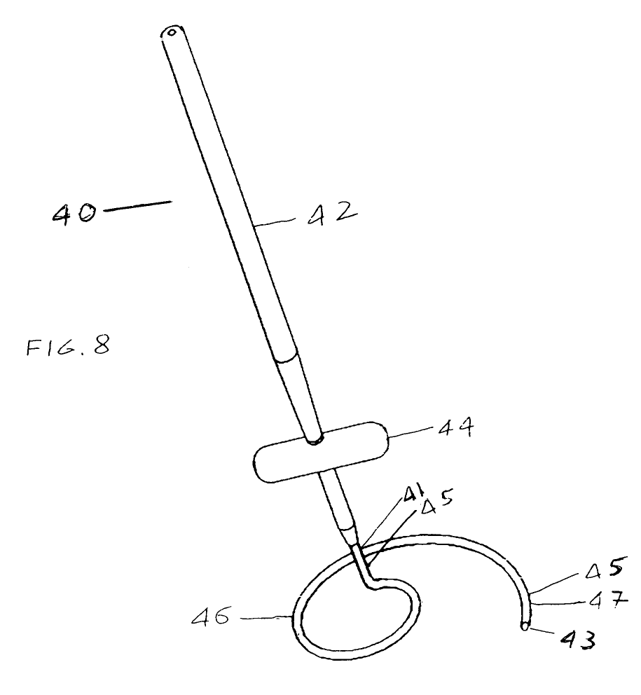

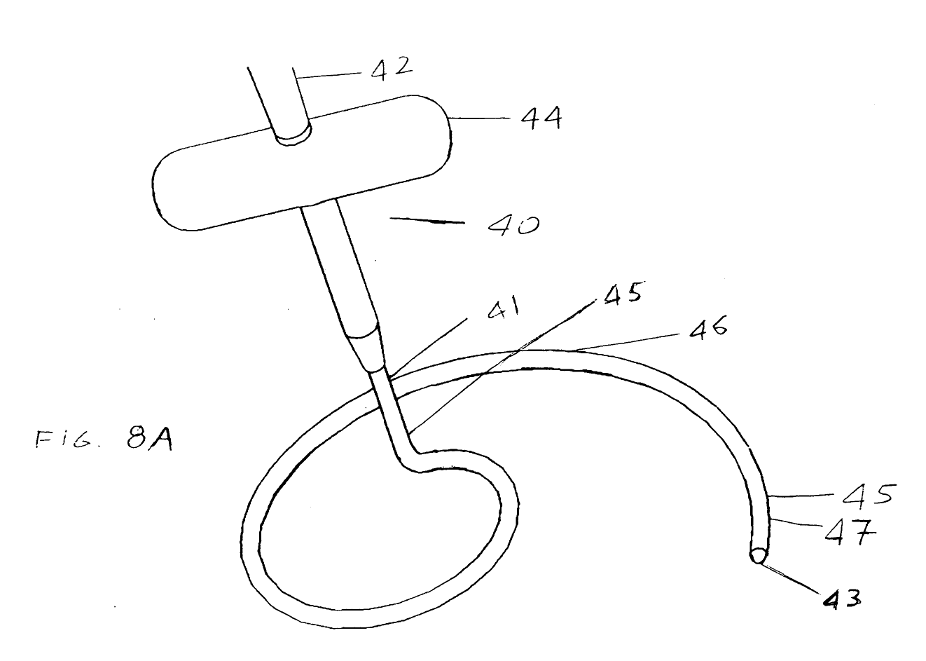

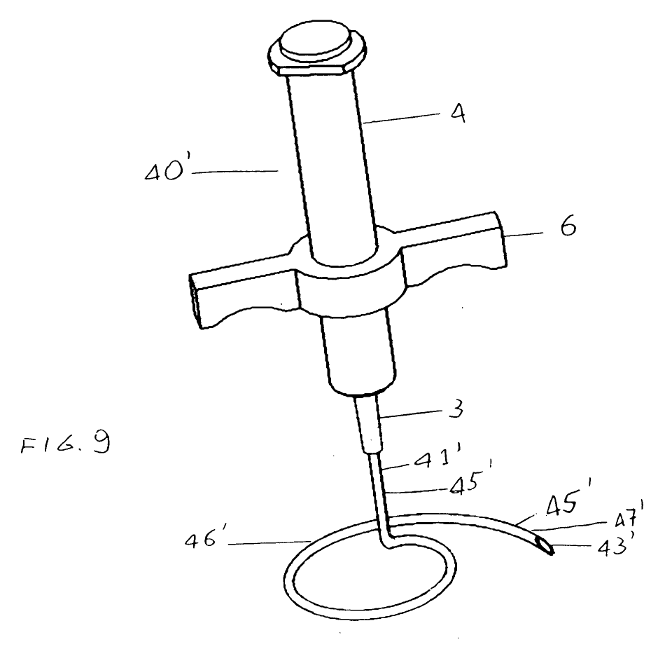

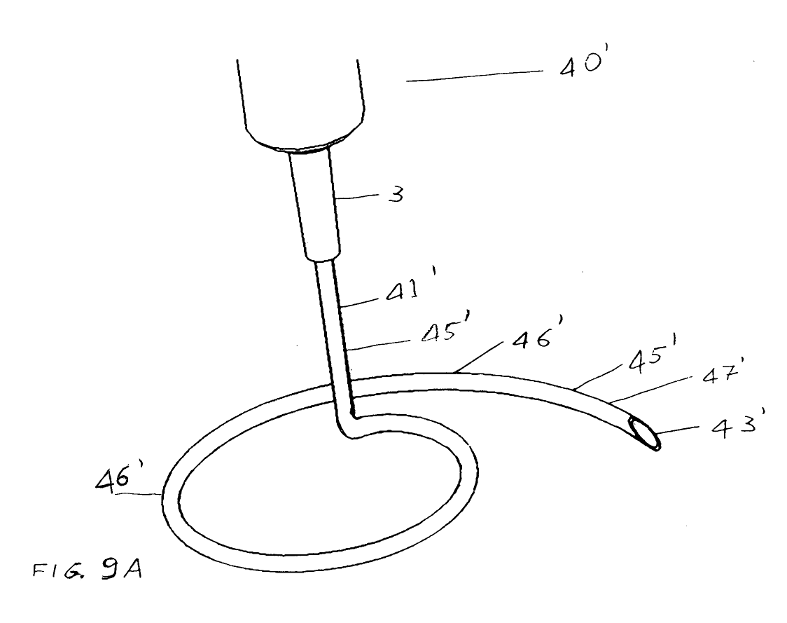

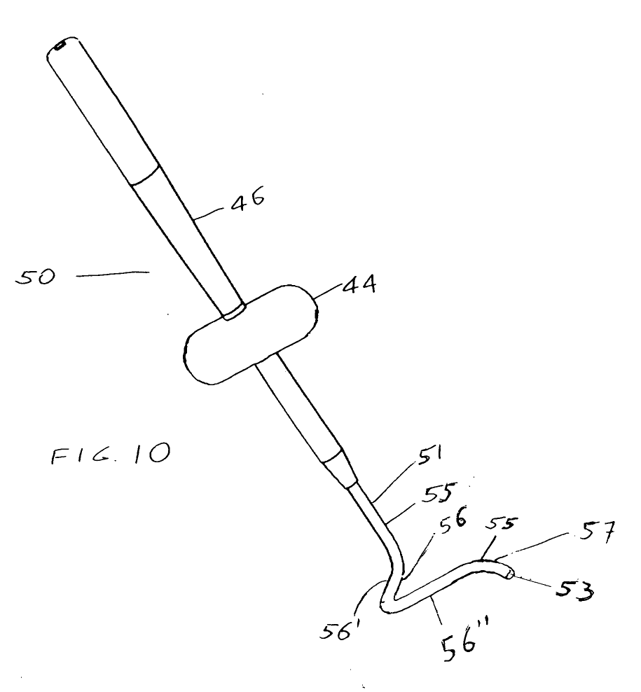

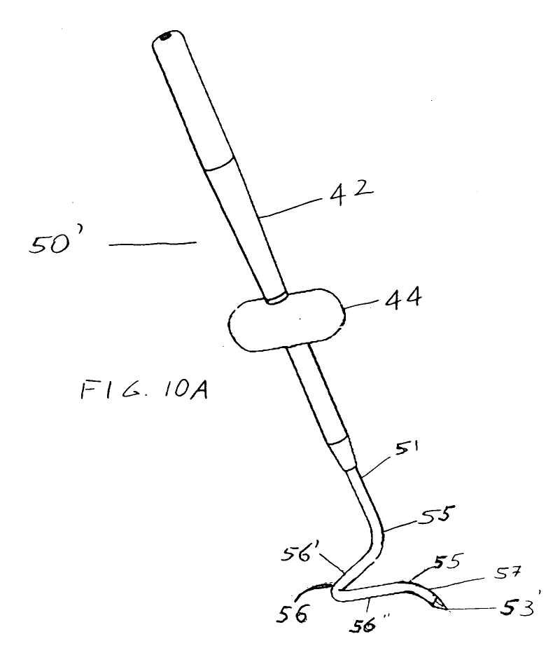

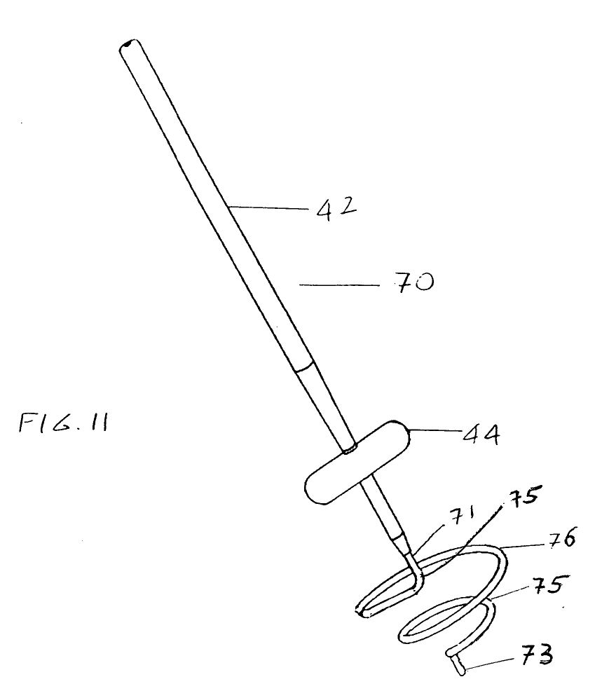

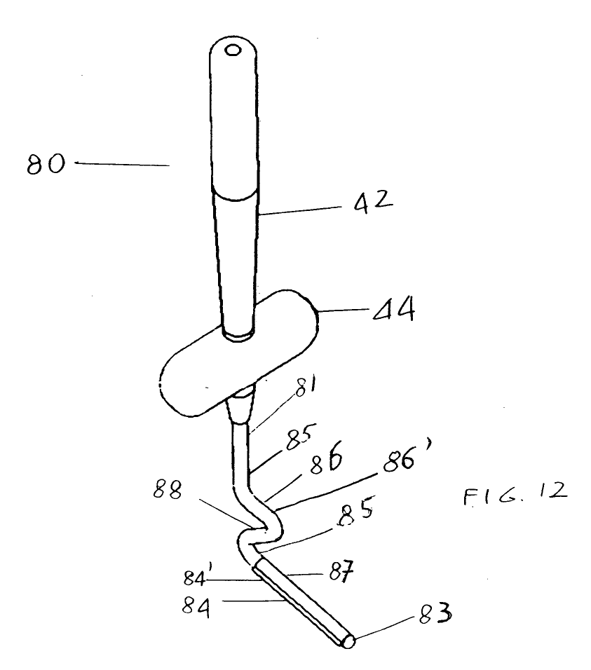

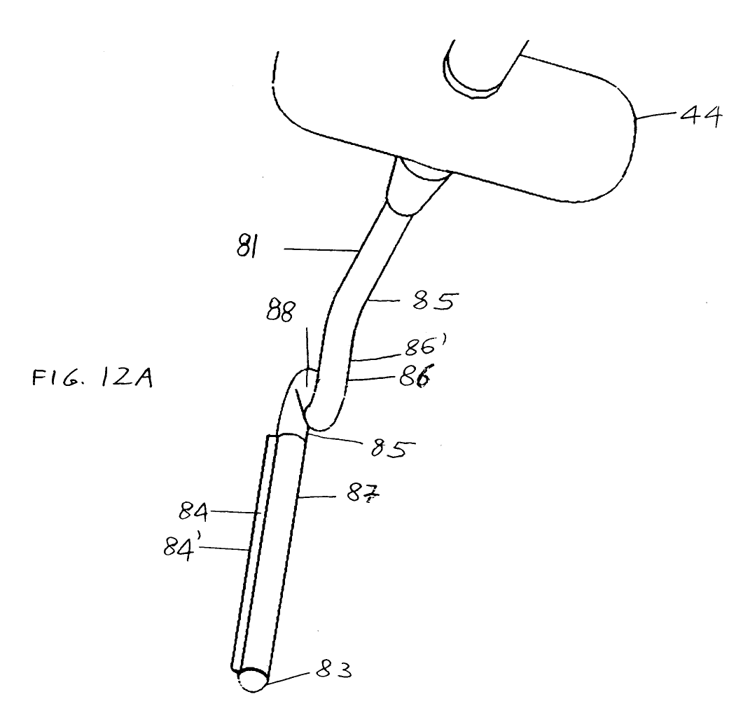

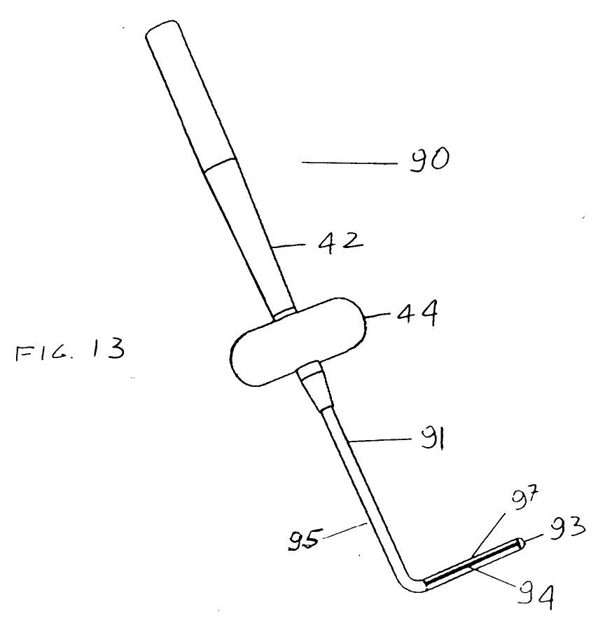

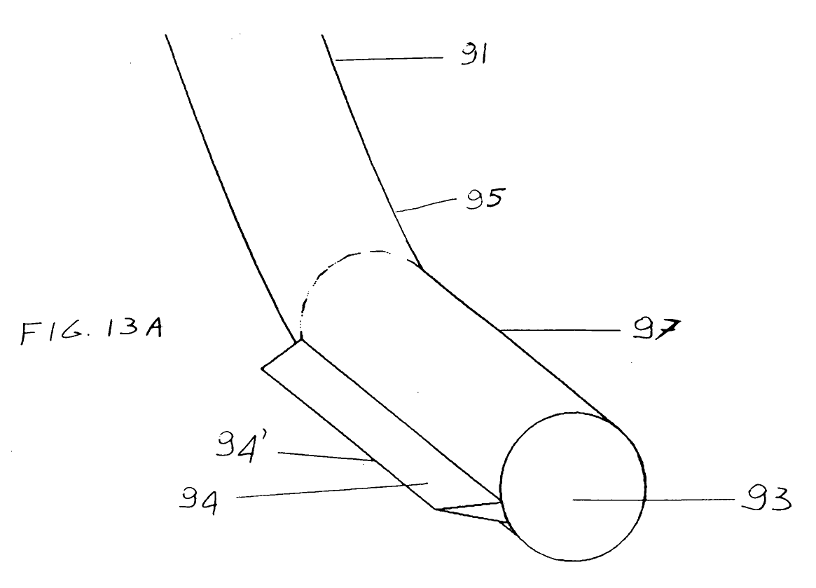

As shown in FIG. 1, Infra-epidermic Subcision Device for Blunt Dissection of Sub-epidermic Tissues or Skin blunt Dissector/Elevator 1 consists of hollow hypodermic needle or skin penetrating means 2 sufficiently rigid to allow skin perforation connected to and in flow communication with syringe or inflating means 4. Needle 2 is in tight sealing connection with syringe 4 via detachable hub 3. Needle tip segment 3′ of needle 2 is imperforated as better shown in FIG. 4, while the remaining segment 3″ of the needle is hollow. Syringe 4 is formed with barrel 8, slideable piston or plunger 10 and handle or handling means or traction or pulling means 6. Syringe is formed at its proximal end with plunger locking mechanism 9 formed with flanges 9′ for the release of locking mechanism 9.

As shown in FIG. 1, balloon or expandable member or bluntly dissecting member or anchoring member 14, grossly donut shaped once inflated as shown in FIGS. 2 and 3, 4 and 6 is mounted on needle shaft 12 of needle 2.

As better shown in shown in FIG. 4 which is a blown up cross sectional view of needle 2 distal segment, balloon 14, shown inflated, is in flow communication with hollow needle 2 via needle holes or needle perforations 20. Needles holes 20 are proximal to imperforated needle tip 3′. Balloon 14 of FIG. 1,2,4,6 or balloon 14′ of FIG. 3, made of extensible material up to a maximum point of expansion, is sealingly attached to needle shaft 12 via cylindrically shaped balloon extensions or sleeve 22 and 22′ as better shown in FIGS. 1 and 4.

Needle 2 can be formed with different sizes balloons allowing variable radial balloon expansions.

FIG. 3 shows device 1 with larger diameter balloon 14′ for radial-lateral blunt dissection/disruption of cellulitic fibrous bands.

As better shown in FIG. 5, plunger locking members or mechanism 9 of plunger 10 is releasable upon pressing down on flanges 9′ which disengage locking members 9 from plunger 10.

As shown in FIG. 1, plunger 10, at rest prior to use, is withdrawn to a degree just sufficient to fully inflate balloon 14 once plunger 10 is fully downwardly displaced.

As it can be better understood from FIG. 6, which shows the device in use, the operator advances needle 2 with imperforated tip 3′ into the patient skin 30. Local anesthetic can be administered prior to skin insertion of needle tip 3′ for pain relief. Needle 2 is preferably inserted in the depressed center of a skin dimple 21′ of the cellulitic skin 30. Dimple 21′ is shown before skin traction, while dimple 21 is shown in FIG. 6 during skin traction, as it will be described below. Once needle tip 3′ and distal segment of needle shaft 12 with balloon 14 is at sufficient depth underneath the epidermis, balloon 14 is inflated by the operator by advancement of plunger 10. Upon full advancement, plunger 10 is locked by locking mechanism 9 in its fully advanced position, as shown in FIGS. 2, 3 and 6. Upon full advancement of plunger 10, balloon 14 inflates and expands radially-laterally. Radial-lateral expansion of balloon 14 and to a larger degree of balloon 14′ of larger diameter, will stretch cellulitic fibrous bands 24 to a point of rupture, via blunt dissection or disruption. Cellulitic fibrous bands are shown in FIG. 6 before blunt disruption 24′ and after disruption at 24. When fully expanded, balloons 14 or 14′ act as sub-epidermic anchoring device for skin traction. The operator pulls the device away from the skin surface via handle or traction means 6. Balloon or expandable member or bluntly dissecting member or anchoring member 14 or 14′ grossly donut shaped, sub-epidernically placed indeed act as anchoring member allowing elevation/traction of the skin. By elevating the skin, fibrous bands 24 are bluntly disrupted and dissected from attachments to epidermis 25 or from attachments to the deeper skin layers 25′, as shown in FIG. 6. Skin dimples 21, no longer tethered down by fibrous bands 24 and or 24′ will be free to rise by natural resiliency to the level of the surrounding skin.

The operator can repeat the procedure by inserting the needle into each cellulitic skin dimple 21′. By operating the device as described, the operator can eliminate, one by one, every skin dimple, restituting normal appearance to the skin.

FIG. 7 shows another embodiment of device 1 of FIG. 1-6, generally indicated at 29. Device 29 is in all similar to device 1 of FIG. 1-6 except that hvpodermic needle or elongated member 34 is mounted with coaxial catheter or flexible sleeve 36 formed with balloon or expandable member 35. Catheter 36 is sealinglv connected via hub 33 to hub 3 of needle 34. Needle 34 is formed with entry segment 34′, L-shaped, provided with tip 31 and dissecting means or blade 39 as better shown in FIG. 7A. Tip 33 of entry segment 34′ is shown blunt in FIG. 7A, but can be also sharp to allow skin penetration.

As better shown in FIG. 7B which is a cross sectional view of balloon 35, of catheter 36 and of hollow needle 34, hollow needle 34 is longitudinally fenestrated via longitudinal opening 37. Catheter 36 is tightly mounted over needle 34 and is provided with openings 38 which are aligned, and in flow communication, with opening 37 of needle 34.

As seen in FIG. 7B, distal segment 36′ of catheter 36 extends into an enlarged and/or expandable segment of such catheter, balloon 35, whose wall or distensible airtight membrane 35′ is folded over catheter shaft 36″ and, as best seen in FIG. 7C, it extends over proximal segment 32 of catheter 36 reducing its diameter into sleeve 32′, which is sealingly bound over proximal segment 32 of catheter 36.

This version offers manufacturing advantages over versions where balloon is sealingly bound with adhesives over the needle, because in this version no adhesive binding is necessary between needle on one side and catheter/balloon on the other side. In fact, in use, air or fluidous component is delivered, by advancement of plunger 10, from syringe 4 into needle 34 which is hollow up to its fenestration 37. Air will preferentially select the pathway of least resistance, and will enter balloon 35 via openings 37 of needle 34, then via catheter openings 38 which are aligned in flow communication with needle opening 37, rather than opening its way and escaping along the interface between tightly adherent catheter shaft 36 and needle 34. Upon air build up within balloon 35, consequent pressure build up within balloon 35 will result in increased adherence of catheter shaft 36″ to needle 34, which in turn will prevent escape of air between catheter and needle. Inflated balloon 35 will retain needle 34 from exiting out of the skin when the operator will pull in direction away from the skin the syringe secured to the needle. This action will result with elevation of the skin to such extent of disrupting the attachment of the collagen fibers to the dermis and releasing the skin dimples which characterize the cellulite.

FIG. 8 through 17 illustrate other embodiments of device 1 of FIG. 1-6. Despite varying in structure and design, all these apparatuses have the common denominator of being provided, once introduced into the skin of a patient and deployed. as device 1 of FIG. 1 to 6 and 29 of FIG. 7-7C, with a blunt surface contacting the sub-epidermic layers such the dermis or deeper tissue layers allowing traction and elevation of the skin from underneath by the operator.

Elevation of the skin will result in blunt dissection/disruption of the cellulitic fibrous bands at the skin attachment and or at deeper layer attachment such as at attachment on the fascia. Another application of the devices above and below described, in addition to the treatment of cellulite is the treatment of any depressed scar or even deep wrinkles where dissection/disruption of the fibrotic bands from the dermis or deeper attachments, responsible of the scar tissue or deep wrinkles, will result in elevation of the depressed skin surfaces to an even anatomical level with the surrounding skin surface. An example of this application is the correction and cosmetic amelioration of acne scars.

FIG. 8 illustrates device 40 composed of handle 42 generally of elongated shape such as cylindrical or hexagonal, formed with handle bar 44 to result into a generally T-shaped combination, and entry segment 47 with dermis or blunt skin lifting segment or sub-epidermic contacting member spirally shaped 46 formed with blunt tip 43 as shown in FIG. 8 and 8A.

Elongated member 45 of device 40 can be made of a substantially rigid material such as medical grade steel allowing penetration and manipulation of the device by handle 42. Elongated member 45 is composed of a stem member 41 and of an arm or lifting means 46 having a blunt surface.

Handle 42 and handle bar 44 can be made of any suitable material including plastic.

The device can be made disposable mono-use or re-sterilizable multi-use.

In operation the skin of a patient is punctured with an ordinary hypodermic needle after proper skin prepping and eventually the skin area is infiltrated with a local anesthetic. Blunt tip 43 of device 40 is then inserted into the skin opening created by the hypodermic needle tip. The operator then rotates device 40 in a clockwise fashion by acting upon handle 42 and handle bar 44 allowing full penetration of entrv segment 46 underneath the skin.

Once spiral segment or dermis blunt lifting segment 46 is well positioned underneath the skin, the operator will pull upward device 40. In doing so the cellulitic fibrotic bands present in the dermis as described for device 1 of FIG. 1-6 or for device 29 of FIG. 8-8B will be severed bv traction exerted perpendicularly to the surface of the skin by maintaining the longitudinal axis of the handle oriented perpendicularly to the surface of the skin. The skin will be lifted as dermis lifting segment 46 provides a blunt dermis contacting surface from underneath the skin for skin lifting purposes.

FIGS. 9 and 9A illustrates device 40′ which, as device 1 of FIG. 1-6, is composed of syringe 4 to which hollow needle or elongated member 45′ is sealingly connected via hub 3. Needle 45′ is formed with spirally shaped entry segment 47′ and blunt skin lifting member 46′. Elongated member 45′ is hollow, in flow communication with syringe 4 and formed with sharp tip 43′. Device 40′ is used as device 40 except that, being tip 43′ sharp, it allows penetration and placement of dermis or skin blunt lifting segment 46′ underneath the skin without prior use of an hypodermic needle for creating a skin opening, as needed for described device 40. Syringe 4 can be pre-filled with any type of medication that the operator believes is suitable to be delivered into the dermis, subcutaneous tissue or into deeper tissues, including anesthetics. and lipolytic or in general tissue-lysing medications such as, for instance, the enzyme collagenase.

FIG. 10 illustrates device 50 in all similar to device 40 of FIGS. 8 and 8A in structure, use and operation with the difference that elongated member 55 is double L-shaped with tip 53 being blunt. Blunt lifting member is indicated at 56. With the longitudinal axis of the device being oriented vertically, the first L is oriented on a vertical plane, and composed of vertical segment or stem member 51 and horizontal segment 56′, the second L, is oriented on an horizontal plane and is composed of horizontal segments 56′ and 56″.

FIG. 10A illustrates device 50′ in all similar to device 50 of FIG. 10 in structure, use and operation with the difference that double L shaped elongated member 55 is formed with tip 53′ being sharp.

Device 50 and 50′ are operated as device 40 of FIG. of FIGS. 8 and 8A and 40′ of FIGS. 9 and 9A.

To aid fibrous bands detachments being already accomplished by axial upward traction, the operator, beside lifting the skin as already described, can rotate the device by acting upon handle 42, and handle bar 44. Rotation of elongated member 55 will dissect any tissue fibrotic attachment met during the rotation.

FIG. 11 illustrates device 70, in all similar to device 40 of FIG. 8-8A in use and operation, with the difference that elongated member 75 is composed of stem member 71 and arm or lifting means 76 helicoidally shaped. Tip 73 of elongated member 75 can be either blunt as illustrated in FIG. 11 or sharp.

The device is operated as device 40 of FIGS. 8 and 8A and actually is screwed into the skin as a corkscrew into a cork.

FIG. 12 shows another embodiment of device 40 of FIG. 8-8A, generally indicated at 80 in all similar to device 40 in use and operation except that elongated member 85 is grossly Z shaped. As better shown in FIG. 12A, skin lifting or sub-epidermic contacting member 86 is formed with blunt lifting arm or blunt dermis-contacting arm or member 86′, connected via arm 88 to dissecting arm or entry member 87 formed with dissecting blade 84 having edge 84′, which can be either sharp blunt or teethed.

Tip 83 of elongated member 85 is shown blunt but can also be sharp as for the previously described devices.

Once elongated member 85 is inserted and placed under the skin, and skin lifting or sub-epidermic contacting member 86 is below, or within, the dermis, the operator can pull device 80 upwardly via handle 42. As a result of the traction exerted on the device, the skin will be also placed under traction by blunt lifting arm or blunt dermis-contacting arm or member 86 engaging the undersurface of the dermis or the inside of the dermis.

The operator can facilitate or promote detachment of the fibrous bands by imparting rotation to the device by acting upon handle 42 and handle bar 44. As a result of such rotation, dissecting arm 87 with blade 84 will rotate and, consequently, will sharply or bluntly dissect the fibrous bands attached to the skin, such as fibrotic bands characterizing the skin depressions of cellulite or acne scars, while blunt skin lifting arm 86 will keep the dermis or anything above arm 86′ clear from dissection caused bv dissecting arm or member 87. Arm 87 will induce tensile traction on the fibrous bands, enabling blade 84, mounted on dissecting arm 87, to sever such fibrous bands more economically and efficiently than it would be possible without applying tensile traction upon such fibrous bands.

FIG. 13 shows device 90 in all similar to device 40 of FIG. 8-8A in use and operation except that elongated member 95 as better shown in FIGS. 13A and 13B is L shaped and composed of stem member 91 and of skin lifting and dissecting arm 97 formed with dissecting blade 94. being blade edge 94′ sharp or blunt as for device 80 of FIG. 12. Tip 93 is illustrated blunt.

The device is operated as device 80 of FIGS. 12 and 12A.

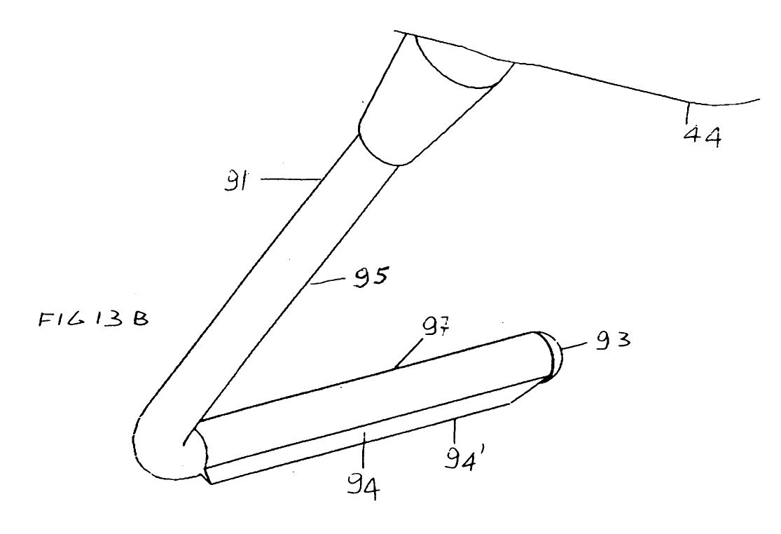

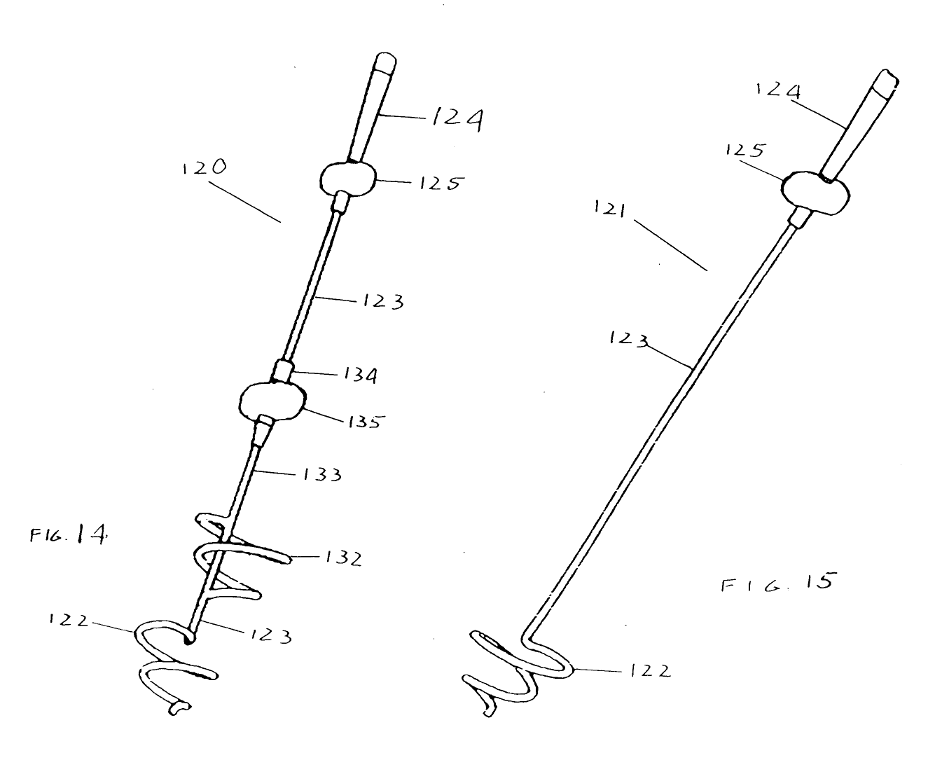

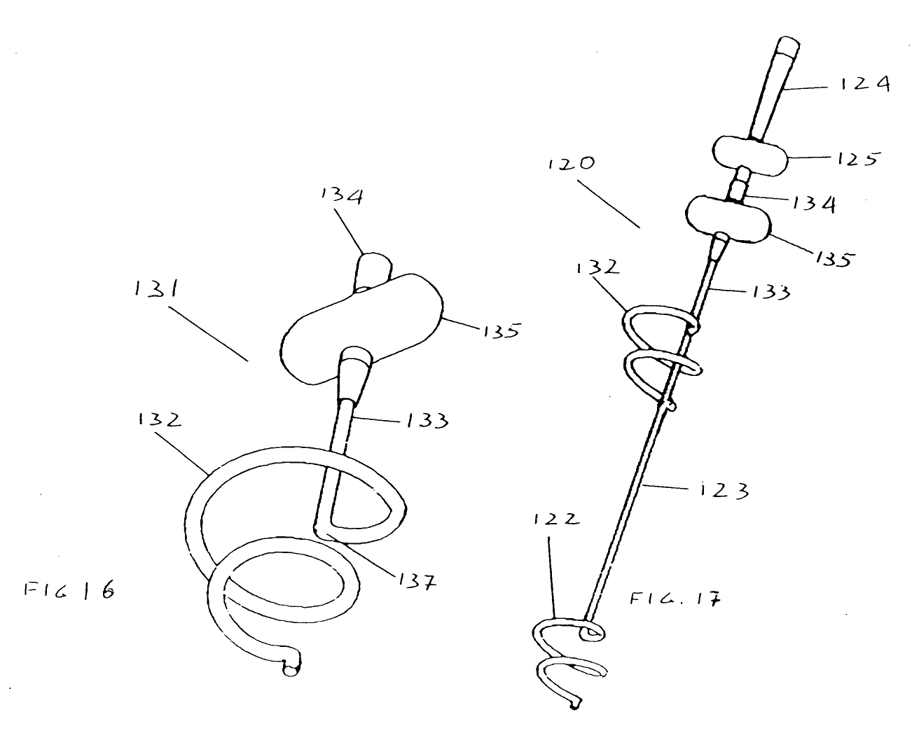

FIG. 14 illustrates device 120. This embodiment has definite similarities with device 70 of FIG. 11. Device 120 has two components, components 121 and component 131. Component 121, as best seen in FIG. 15 is in all similar to device 70 of FIG. 11 with the only significant difference that vertical segment or stem member 123 is much longer than in device 70 of FIG. 11. Handle 124 of component 121 has bar 125 to facilitate rotation of component 121 during use. Helicoidal segment or anchoring means 122 of component 121 is in all similar to helicoidal segment 76 of device 70 of FIG. 11. Component 131, as best seen in FIG. 16 is also similar to device 70 Of FIG. 11 with the significant difference that vertical segment or stem member 133 is hollow, with distal opening 137, and telescopically slides over vertical segment 123 of component 121. Segment 133 of component 131 is connected to handle 134. Handle 134 is also hollow in order to slides over segment 123 of component 121, and has bar 135 to facilitate rotation of component 131 during its use. Helicoidal segment 132 of component 131 is also similar to helicoidal segment 76 of device 70 of FIG. 11. Segment 122 of component 121 and segment 132 of component 131 may have either a sharp tip or a blunt tip. The device is operated by inserting segment 122 of component 121 into the skin. If segment 122 has a sharp tip, segment 122 will be inserted directly into the skin after local anesthesia. If segment 122 has a blunt tip, segment 122 will be inserted into the skin by engaging the blunt tip of segment 122 into a skin hole made with a needle after proper local anesthesia.

Segment 122 will be advanced into the skin bv the operator bv rotating bar 125 of handle 124 which will result with a type of corkscrew advancement of segment 122. When segment 122 has advanced into the subcutaneous tissue and is in proximity of the muscle layer, the operator will engage the tip of segment 132 of component 131 into the same skin hole where segment 122 of component 121 had entered. Segment 132 will be advanced into the skin in the fashion segment 122 is advanced, by rotating bar 135 of handle 134. When segment 132 of component 131 has entered the subcutaneous tissue, the operator will hold handle 134 down on the skin while pulling handle 124 of component 121 away from the skin. This action will result in separating further apart segment 122 from segment 132, as best seen in FIG. 17, and, with them the layers they are engaged with. This embodiment has a clear advantage over all the embodiment described above in the fact that it anchors the attachment of the fibrous bands on the deep layers while it exerts traction on the superficial attachments of the fibrous bands avoiding the possibility that traction exerted upon the superficial attachments of the fibrous bands results into an elevation of the deeper layer rather than in detachment of the fibrous bands.

What we claim is:

1. A minimally invasive skin lifting device for placing traction on an area of a skin, comprising:
skin lifting means placeable by skin puncturing underneath a layer of the skin, said skin lifting means comprising a blunt surface for contacting said layer of the skin from beneath to elevate said layer of the skin upon traction exerted on said lifting means.

2. The device of claim 1, wherein said lifting means placeable by skin puncturing are carried into the skin by an elongated member having substantial rigidity for skin penetration, through a skin hole resulting from said skin puncturing, said skin hole having a diameter, said blunt surface for contacting said layer of the skin from beneath having at least one contacting surface dimension being greater than said diameter of the skin hole.

3. The device of claim 2, further comprising:
handle means to enable the operator to manipulate said elongate member to position said lifting means into the skin beneath a layer of the skin and the exert traction upon said handle to lift said skin lifting means to lift the skin.

4. The device of claim 3, further comprising:
dissecting member for tissue dissection distally mounted to said lifting means to cause tissue dissection by rotation of said elongated member via said handle means

5. The device of claim 2 wherein said skin lifting means comprises an expandable member.

6. The device of claim 5 wherein said expandable member comprises an inflatable member.

7. The device of claim 5 further comprising:
a catheter over said elongated member, said elongated member comprising a rigid needle having a hollow segment and an imperforated tip segment, and said expandable member carried on said elongated member comprising an inflatable member formed from a catheter over said needle, said inflatable member being in flow communication with said hollow segment of said needle, and inflating
means in flow communication with said hollow segment of said needle, causing expansion of said inflatable member after placement of said inflatable member beneath said layer of the skin, to enable lifting of said layer of the skin, said lifting causing detachment of subcutaneous fibrous bands, said expansion causing blunt dissection of tissues laving beneath said superficial layer of the skin.

8. The device of claim 6 wherein:
said inflatable member further comprises a flexible sleeve mounted over said elongated member, said flexible sleeve having a proximal segment, a distal segment and a shaft segment, said distal segment being in continuity with a distendable airtight membrane to enclose said shaft segment and being sealed to said proximal segment of said sleeve to form said inflatable member, said shaft segment having at least one opening,
said elongated member having a hollow segment and an imperforated tip segment, said hollow segment having at least one opening in flow communication with said opening of the shaft segment of the flexible sleeve,
said hollow segment of said elongated member being in flow communication with a fluidous component delivery system
said inflatable member being inflatled by said fluidous component delivery system to a pressure sufficient to seal said shaft segment of said flexible sleeve upon said hollow segment of said elongated member, and sufficient to form said blunt skin contacting surface sufficiently rigid, in use, to enable lifting of the skin.

9. The device of claim 2 wherein said elongated member comprises a stem member that upon lifting of the skin is held substantially perpendicular to the surface of the skin and wherein said lifting means is shaped as an arm stemming at an angle from a distal end of said stem member, said arm, upon lifting of the skin, being oriented substantially parallel to the surface of the skin.

10. The device of claim 9 wherein said said arm stemming at an angle from said stem member is substantially straight and is formed with means for sharp dissection of subcutaneous tissue by rotation of said arm pivoting on said stem member.

11. The device of claim 9 wherein said arm stemming at an angle from a distal end of said stem member is bent.

12. The device of claim 11 wherein said arm stemming at an angle from said stem member is formed with dissecting means dissecting tissues by rotation.

13. The device of claim 11 wherein said arm stemming at an angle from said stem member is spirally shaped.

14. The device of claim 13 wherein said said arm stemming at an angle from said stem member is in flow communication with a syringe allowing delivery of medications into skin layers.

15 The device of claim 11 wherein said said arm stemming at an angle from said stem member is helicoidally shaped.

16. The device of claim 9, further comprising anchoring means slideably mounted over said stem member, said anchoring means engaging a deeper layer of the skin to anchor subcutaneous tissue and allow disruption of subcutaneous tissue upon lifting of the skin via traction exerted upon said lifting means.
